(19) United States
(12) Patent Application Publication
(10) Pub. No.: US 2015/0065380 A1
(43) Pub. Date: Mar. 5, 2015

(54) Brain somatic mutations associated to epilepsy and uses thereof

(71) Applicants: Korea Advanced Institute of Science and Technology, Daejeon (KR); Yonsei University, University - Industry Foundation (UIF), Seoul (KR)

(72) Inventors: Jeong Ho LEE, Daejeon (KR); Dong Seok Kim, Seoul (KR); Hoon Chul Kang, Seoul (KR); Jae Seok Lim, Daejeon (KR); Woo-II Kim, Daejeon (KR)

(21) Appl. No.: 14/474,596

(22) Filed: Sep. 2, 2014

(30) Foreign Application Priority Data
Sep. 3, 2013 (KR) 10-2013-0105712
Jun. 12, 2014 (KR) 10-2014-0071588

(51) Int. Cl. G01N 33/573; C12N 9/12; C12Q 1/68; C07K 16/40

(52) U.S. Cl. CPC G01N 33/573; C07K 16/40; C12N 9/12; C12Q 1/6883; C12Y 207/11001; G01N 2800/2857; C12Q 2600/156; C12Q 2600/16; G01N 2333/91205
USPC 506/9; 435/7.4; 536/23.2; 530/387.9; 536/24.33; 536/24.31; 536/24.5; 435/194; 435/6.11

(57) ABSTRACT

The present invention relates to epilepsy-inducing brain somatic mutations which are associated with intractable epilepsy caused by malformations of cortical development, and uses thereof. More particularly, the present invention relates to an mTOR (Mammalian target of rapamycin) gene having mutations in a nucleotide sequence or an mTOR protein having mutations in an amino acid sequence resulting from the mutations in the nucleotide sequence. Further, the present invention relates to a technique for diagnosing intractable epilepsy caused by malformations of cortical development using the gene or the protein.

### CROSS-REFERENCE TO RELATED APPLICATIONS

This application claims the benefit of Korean Patent Application No. 10-2013-0105712 on Sep. 3, 2013, and 10-2014-0071588 on Jun. 12, 2014 with the Korean Intellectual Property Office, the disclosure of which are herein incorporated by reference in its entirety.

### BACKGROUND

1. Field

The present invention relates to epilepsy-inducing brain somatic mutations which are associated with intractable epilepsy caused by malformations of cortical development, and uses thereof. More particularly, the present invention relates to an mTOR (Mammalian target of rapamycin) gene having mutations in a nucleotide sequence or an mTOR protein having mutations in an amino acid sequence resulting from the mutations in the nucleotide sequence. Further, the present invention relates to a technique for diagnosing intractable epilepsy caused by malformations of cortical development using the gene or the protein.

2. Description of the Related Art

Epilepsy is a chronic disease to have recurrent seizures which occur as a result of a sudden excessive electrical and synchronized discharge in brain, and is a severe neurological disease accompanied with neurobiological, psychiatric, cognitive, or social impairments.

Epilepsy is one of the most common neurological diseases, affecting approximately 0.5%-1% of the world population. Worldwide, about 45 new epileptic patients per one hundred thousand people are generated every year. In the USA, it is estimated that there are more than 3 million patients with epilepsy, and about 500 new epileptic patients are reported to be generated every day. Further, 70% of cases of epilepsy begin during childhood or adolescence, and in particular, infants are more likely to have epilepsy. The highest incidence and prevalence rates are observed in the first year after the birth of a child, and then drop rapidly. The incidence and prevalence rates rise rapidly again in people over the age of 60, and thus tend to exhibit a U-shaped curve. The prevalence rate of patients who have experienced epileptic seizures in their lives reaches 10-15%.

Epilepsy that fails to respond to anti-epileptic drugs developed until now is called intractable epilepsy, which accounts for approximately 20% cases of epilepsy worldwide.

Malformations of cortical development (MCD) are one of the most common cause of intractable epilepsy. MCDs are a group of disorders characterized by abnormal development of the cerebral cortex due to abnormalities in neuronal migration, differentiation and proliferation, and cause many neurological comorbidities such as developmental delays, mental retardation and cognitive impairments as well as epilepsy. With recent technological advances in brain imaging, such as high-resolution magnetic resonance imaging, etc., diagnosis of malformations of cortical development in patients with intractable epilepsy is rapidly increasing.

At present, malformations of cortical development are known to be observed in 50% or more of childhood patients with intractable epilepsy that cannot be controlled with medication and thus should be considered for epilepsy surgery. Malformations of cortical development (sporadic MCD) found in childhood patients may occur in one twin of an identical twin pair, and it is also known that sporadic malformations of cortical development occur without specific family history and external stimulation. Understanding of etiology and pathogenetic mechanisms thereof is insufficient.

Depending on clinical and histopathological features, there are several types of malformations of cortical development. Of them, the most frequent focal cortical dysplasia (FCD), hemimegalencephaly (HME) and tuberous sclerosis complex (TSC) do not respond to existing anti-epileptic drugs, and thus neurosurgical treatment to remove brain lesions is required for controlling epilepsy.

Accordingly, there is an urgent need to define the molecular genetic etiology and develop a diagnostic technique specific to malformations of cortical development which cause intractable epilepsy.

### SUMMARY

An aspect provides an isolated protein consisting of an amino acid sequence which includes one or more mutations selected from the group consisting of
substitution of tyrosine (Y) for cysteine (C) at position 1483,
substitution of arginine (R) for cysteine (C) at position 1483,
substitution of lysine (K) for glutamic acid (E) at position 2419,
substitution of glycine (G) for glutamic acid (E) at position 2419,
substitution of proline (P) for leucine (L) at position 2427, and
substitution of glutamine (Q) for leucine (L) at position 2427
in an amino acid sequence of SEQ ID NO. 2.

Another aspect provides a composition including the protein; or an antibody or aptamer specifically binding to the protein.

Still another aspect provides a method for diagnosing intractable epilepsy due to malformations of cortical development, including the steps of treating a sample of a patient with the antibody or aptamer specifically binding to the protein so as to detect the presence of the protein; and determining that the patient has intractable epilepsy due to malformations of cortical development when the protein is detected in the sample of the patient.

Still another aspect provides an isolated gene consisting of a nucleotide sequence which includes one or more mutations selected from the group consisting of
substitution of adenine (A) for guanine (G) at position 4448,
substitution of cytosine (C) for thymine (T) at position 4447,
substitution of adenine (A) for guanine (G) at position 7255,
substitution of guanine (G) for adenine (A) at position 7256,
substitution of cytosine (C) for thymine (T) at position 7280, and
substitution of adenine (A) for thymine (T) at position 7280
in a nucleotide sequence of SEQ ID NO. 1.

Still another aspect provides a composition including the gene; or a primer, probe, or antisense nucleic acid complementarily binding to the gene.

Still another aspect provides a method for diagnosing intractable epilepsy due to malformations of cortical development, including the steps of treating a sample of a patient with the primer, probe, or antisense nucleic acid complementarily binding to the gene so as to detect the presence of the gene; and determining that the patient has intractable epilepsy due to malformations of cortical development when the gene is detected in the sample of the patient.

### BRIEF DESCRIPTION OF THE DRAWINGS

FIG. 1 shows preoperative and postoperative magnetic resonance images and pathologic images of patients with malformations of cortical development (each patient according to the type of diseases is indicated by FCD6, TSC2, and HME1), in which “Pre-op MRI” indicates preoperative magnetic resonance images of patients with malformations of cortical development, “Post-op MRI” indicates postoperative magnetic resonance images of patients with malformations of cortical development, and “Pathology” indicates pathologic images.

FIG. 2 shows preoperative and postoperative magnetic resonance images and pathologic images of patients with malformations of cortical development (each patient is indicated by FCD3, FCD4, and FCD7), in which “Pre-op MRI” indicates preoperative magnetic resonance images of patients with malformations of cortical development, “Post-op MRI” indicates postoperative magnetic resonance images of patients with malformations of cortical development, and “Pathology” indicates pathologic images.

FIG. 3 shows algorithms to search for brain-specific genetic mutations using Virmid (Genome Biology, 14 (8), R90, 2013) and MuTect software (Nature Biotechnology, 31, 213-219 (2013)) at the same time with respect to the results of whole exome sequencing.

FIG. 4 shows percentages of mTOR gene mutations found in the patients with malformations of cortical development and genetic mutations found in the brain tissues.

FIG. 5 shows genetic mutations detected in the mTOR target site (containing amino acids, Cys1483, Glu2419, and Leu2427) in the brain tissues of 76 patients with focal cortical dysplasia type IIa (FCDIIa) and focal cortical dysplasia type IIb (FCDIIb), and mutations rates thereof (%).

FIG. 6 shows genetic mutations detected in the mTOR target site (containing amino acids, Cys1483, Glu2419, and Leu2427) in the saliva samples of 30 patients with focal cortical dysplasia type IIa and IIb, and mutations rates thereof (%).

FIG. 7 shows the results of Western blot for analyzing S6 phosphorylation in HEK293T cells which were introduced with the wild-type mTOR protein or each of 6 types of mTOR mutants, in which “Empty” indicates HEK293T cells transfected with empty flag-tagged vector, “P-S6” indicates phosphorylated S6 protein, “S6” indicates S6 protein, “Flag” indicates flag protein, and “20% serum” indicates those exposed to 20% serum for 1 hour and is used as a positive control showing the increased mTOR activity.

FIG. 8 shows the results of measuring mTOR kinase activity in HEK293T cells which were introduced with the wild-type mTOR protein or each of 6 types of mTOR mutated proteins (*p<0.05 and ***p<0.001, Error bars, s.e.m.).

### DETAILED DESCRIPTION

In the present invention, each 6 types of mTOR gene mutations which are specifically found in the brain tissues of patients with intractable epilepsy due to malformations of cortical development and mTOR protein mutations thereby were identified (Table 1).

TABLE 1

| | mTOR gene mutations | mTOR protein mutations |
|---|---|---|
| 1 | T4447C | C1483R |
| 2 | G4448A | C1483Y |
| 3 | G7255A | E2419K |
| 4 | A7256G | E2419G |
| 5 | T7280C | L2427P |
| 6 | T7280A | L2427Q |

T4447C indicates a mutation of substitution of cytosine (C) for thymine (T) at position 4447 in nucleotide sequence of mTOR.
G4448A indicates a mutation of substitution of adenine (A) for guanine (G) at position 4448 in nucleotide sequence of mTOR.
G7255A indicates a mutation of substitution of adenine (A) for guanine (G) at position 7255 in nucleotide sequence of mTOR.
A7256G indicates a mutation of substitution of guanine (G) for adenine (A) at position 7256 in nucleotide sequence of mTOR.
T7280C indicates a mutation of substitution of cytosine (C) for thymine (T) at position 7280 in nucleotide sequence of mTOR.
T7280A indicates a mutation of substitution of adenine (A) for thymine (T) at position 7280 in nucleotide sequence of mTOR.
C1483R indicates a mutation of substitution of arginine (R) for cysteine (C) at position 1483 in amino acid sequence of mTOR.
C1483Y indicates a mutation of substitution of tyrosine (Y) for cysteine (C) at position 1483 in amino acid sequence of mTOR.
E2419K indicates a mutation of substitution of lysine (K) for glutamic acid (E) at position 2419 in amino acid sequence of mTOR.
E2419G indicates a mutation of substitution of glycine (G) for glutamic acid (E) at position 2419 in amino acid sequence of mTOR.
L2427P indicates a mutation of substitution of proline (P) for leucine (L) at position 2427 in amino acid sequence of mTOR.
L2427Q indicates a mutation of substitution of glutamine (Q) for leucine (L) at position 2427 in amino acid sequence of mTOR.

Therefore, the present invention provides novel mTOR mutated genes and mTOR mutated proteins thereby. Further, the present invention provides a technique for diagnosing epilepsy by detecting the mutated gene or the mutated protein. Furthermore, the present invention provides a technique for inducing epilepsy by introducing the mutated gene and/or the mutated protein into a cell or an individual.

As used herein, the term “epilepsy” refers to a chronic disease to have recurrent seizures which occur as a result of a sudden excessive electrical discharge in a group of nerve cells. In the present invention, the epilepsy includes intractable epilepsy. Further, the epilepsy may be epilepsy which is caused by malformations of cortical development (MCD), and more preferably, intractable epilepsy which is caused by malformations of cortical development. Further, the malformations of cortical development may be focal cortical dysplasia (FCD), hemimegalencephaly (HME) or tuberous sclerosis complex (TSC). Further, in the present invention, the epilepsy may be epilepsy which is accompanied with gene mutations of mTOR gene or amino acid mutations of mTOR protein.

mTOR (mammalian target of rapamycin) protein is the mammalian target protein of rapamycin, and is known as FK506 binding protein 12-rapamycin associated protein 1 (FRAP1). mTOR protein is expressed by FRAP1 gene in humans. mTOR protein is a serine/threonine protein kinase that functionally regulates cell growth, cell proliferation, cell death, cell survival, protein synthesis and transcription, and belongs to the phosphatidylinositol 3-kinase-related kinase protein family. In the present invention, the wild-type mTOR gene sequence is represented by SEQ ID NO. 1, and the mTOR protein sequence is represented by SEQ ID NO. 2.

As used herein, the term “mTOR mutated gene” means that a mutation occurs in the nucleotide sequence of SEQ ID NO. 1 of the wild-type mTOR gene. Preferably, it may be a gene consisting of a nucleotide sequence which includes one or more mutations selected from the group consisting of substitution of C for T at position 4447, substitution of A for G at position 4448, substitution of A for G at position 7255, substitution of G for A at position 7256, substitution of C for T at position 7280, and substitution of A for T at position 7280 in the nucleotide sequence of SEQ ID NO. 1.

As used herein, the term “mTOR mutated protein” means that a mutation occurs in the amino acid sequence of SEQ ID NO. 2 of the wild-type mTOR protein. Preferably, it may be a protein consisting of an amino acid sequence which includes one or more mutations selected from the group consisting of substitution of R for C at position 1483, substitution of Y for C at position 1483, substitution of K for E at position 2419, substitution of G for E at position 2419, substitution of P for L at position 2427, and substitution of Q for L at position 2427 in the amino acid sequence of SEQ ID NO. 2.

Further, the mTOR mutated protein may include an additional mutation within the scope of not altering generally the molecular activity. Amino acid exchanges in proteins and peptides which do not generally alter the molecular activity are known in the art. In some cases, the mTOR mutated protein may be modified by phosphorylation, sulfation, acrylation, glycosylation, methylation, farnesylation or the like.

In one aspect, the present invention relates to an isolated protein consisting of an amino acid sequence which includes one or more mutations selected from the group consisting of substitution of tyrosine (Y) for cysteine (C) at position 1483, substitution of arginine (R) for cysteine (C) at position 1483, substitution of lysine (K) for glutamic acid (E) at position 2419, substitution of glycine (G) for glutamic acid (E) at position 2419, substitution of proline (P) for leucine (L) at position 2427, and substitution of glutamine (Q) for leucine (L) at position 2427 in the amino acid sequence of SEQ ID NO. 2.

In the protein, the protein having the substitution of tyrosine (Y) for cysteine (C) at position 1483 may be encoded by the gene having substitution of adenine (A) for guanine (G) at position 4448 in the nucleotide sequence of SEQ ID NO. 1,

the protein having the substitution of arginine (R) for cysteine (C) at position 1483 may be encoded by the gene having substitution of cytosine (C) for thymine (T) at position 4447 in the nucleotide sequence of SEQ ID NO. 1,

the protein having the substitution of lysine (K) for glutamic acid (E) at position 2419 may be encoded by the gene having substitution of adenine (A) for guanine (G) at position 7255 in the nucleotide sequence of SEQ ID NO. 1,

the protein having the substitution of glycine (G) for glutamic acid (E) at position 2419 may be encoded by the gene having substitution of guanine (G) for adenine (A) at position 7256 in the nucleotide sequence of SEQ ID NO. 1,

the protein having the substitution of proline (P) for leucine (L) at position 2427 may be encoded by the gene having substitution of cytosine (C) for thymine (T) at position 7280 in the nucleotide sequence of SEQ ID NO. 1, and

the protein having the substitution of glutamine (Q) for leucine (L) at position 2427 may be encoded by the gene having substitution of adenine (A) for thymine (T) at position 7280 in the nucleotide sequence of SEQ ID NO. 1.

In another aspect, the present invention relates to a composition including the protein; or an antibody or aptamer specifically binding to the protein.

The antibody or aptamer may specifically bind to the mutated region of the protein (that is, the region including one or more amino acid residues selected from the group consisting of positions 1483, 2419 and 2427).

Further, the antibody or aptamer is able to specifically detect a mutation in one or more amino acid residues selected from the group consisting of positions 1483, 2419 and 2427 of the protein.

In still another aspect, the present invention relates to a composition for inducing intractable epilepsy due to malformations of cortical development, including the protein.

In still another aspect, the present invention relates to a diagnostic composition for intractable epilepsy due to malformations of cortical development, including the antibody or aptamer specifically binding to the protein.

In still another aspect, the present invention relates to a diagnostic kit for intractable epilepsy due to malformations of cortical development, including the antibody or aptamer specifically binding to the protein.

In still another aspect, the present invention relates to a method for inducing intractable epilepsy due to malformations of cortical development, including the step of introducing the protein into a cell or an individual.

In still another aspect, the present invention relates to a method for diagnosing intractable epilepsy due to malformations of cortical development, including the steps of treating a sample of a patient with the antibody or aptamer specifically binding to the protein so as to detect the presence of the protein; and determining that the patient has intractable epilepsy due to malformations of cortical development when the protein is detected in the sample of the patient.

In still another aspect, the present invention relates to an isolated gene consisting of a nucleotide sequence which includes one or more mutations selected from the group consisting of substitution of adenine (A) for guanine (G) at position 4448, substitution of cytosine (C) for thymine (T) at position 4447, substitution of adenine (A) for guanine (G) at position 7255, substitution of guanine (G) for adenine (A) at position 7256, substitution of cytosine (C) for thymine (T) at position 7280, and substitution of adenine (A) for thymine (T) at position 7280 in a nucleotide sequence of SEQ ID NO. 1.

In still another aspect, the present invention relates to a composition including the gene; or a primer, probe, or antisense nucleic acid complementarily binding to the gene.

The primer, probe, or antisense nucleic acid may complementarily bind to the mutated region of the gene (that is, the region including one or more bases selected from the group consisting of positions 4447, 4448, 7255, 7256 and 7280).

Further, the primer, probe, or antisense nucleic acid is able to specifically detect a mutation in one or more bases selected from the group consisting of positions 4447, 4448, 7255, 7256 and 7280 of the gene.

In still another aspect, the present invention relates to a composition for inducing intractable epilepsy due to malformations of cortical development, including the gene.

In still another aspect, the present invention relates to a diagnostic composition for intractable epilepsy due to malformations of cortical development, including the primer, probe, or antisense nucleic acid complementarily binding to the gene.

In still another aspect, the present invention relates to a diagnostic kit for intractable epilepsy due to malformations of cortical development, including the primer, probe, or antisense nucleic acid complementarily binding to the gene.

In still another aspect, the present invention relates to a method for inducing intractable epilepsy due to malformations of cortical development, including the step of introducing the gene into a cell or an individual.

In still another aspect, the present invention relates to a method for diagnosing intractable epilepsy due to malformations of cortical development, including the steps of treating a sample of a patient with the primer, probe, or antisense nucleic acid complementarily binding to the gene so as to detect the presence of the gene; and determining that the patient has intractable epilepsy due to malformations of cortical development when the gene is detected in the sample of the patient.

The antibody or aptamer specifically binding to the mTOR mutated protein provided in the present invention may be used for detecting the mTOR mutated protein in a sample of a patient. In one specific embodiment, the antibody or aptamer may be an antibody or aptamer specifically binding to the protein consisting of an amino acid sequence which includes one or more mutations selected from the group consisting of substitution of R for C at position 1483, substitution of Y for C at position 1483, substitution of K for E at position 2419, substitution of G for E at position 2419, substitution of P for L at position 2427, and substitution of Q for L at position 2427 in the amino acid sequence of SEQ ID NO. 2.

The antibody or aptamer may specifically bind to the mutated region of the protein, that is, the region including one or more amino acid residues selected from the group consisting of positions 1483, 2419 and 2427. Further, the antibody or aptamer is able to specifically detect a mutation in one or more amino acid residues selected from the group consisting of positions 1483, 2419 and 2427 of the protein. Preferably, the antibody may be a monoclonal antibody or a polyclonal antibody.

The term “antibody”, is known in the art, refers to a specific protein molecule that is directed by an antigenic region. With respect to the objects of the present invention, the antibody means an antibody specifically binding to the mTOR mutated protein which is a marker of the present invention. To prepare the antibody, the mTOR mutated gene is cloned into an expression vector according to the typical method, so as to obtain the mTOR mutated protein encoded by the mTOR mutated gene, and then the antibody may be prepared from the obtained mTOR mutated protein according to the typical method, in which a partial peptide prepared from the mTOR mutated protein is also included, and the partial peptide of the present invention includes at least 7 amino acids, preferably 9 amino acids, and more preferably 12 amino acids or more. There is no limitation in the form of the antibody of the present invention, and a polyclonal antibody, a monoclonal antibody, or a part thereof having antigen-binding property is also included in the antibody of the present invention, and all immunoglobulin antibodies are included. Furthermore, specialized antibodies such as humanized antibody are also included in the antibody of the present invention.

The antibody used for the detection of a diagnostic biomarker for epilepsy of the present invention includes complete forms having two full-length light chains and two full-length heavy chains, as well as functional fragments of antibody molecules. The functional fragments of antibody molecules refer to fragments retaining at least an antigen-binding function, and include Fab, F(ab′), F(ab′)2, Fv and the like.

As used herein, the term “aptamer” refers to a nucleic acid molecule having a binding affinity for a particular target molecule. The aptamer of the present invention may be an RNA, a DNA, a modified nucleic acid or a mixture thereof, which can also be in a linear or circular form. The aptamers, like peptides generated by phage display or monoclonal antibodies, are capable of specifically binding to selected targets. A typical aptamer is 10-15 kDa in size (30-45 nucleotides), and binds to its target with sub-nanomolar affinity. Aptamers are capable of binding to the selected targets through binding interactions (e.g., hydrogen bonding, electrostatic complementarities, hydrophobic contacts, steric exclusion) or specificity in antibody-antigen complexes.

The primer, probe, or antisense nucleic acid that complementarily binds to the mTOR mutated gene provided in the present invention can be used to detect the mTOR mutated gene in a sample of a patient. Preferably, the primer, probe or antisense nucleic acid specifically binds to the gene consisting of a nucleotide sequence which includes one or more mutations selected from the group consisting of substitution of C for T at position 4447, substitution of A for G at position 4448, substitution of A for G at position 7255, substitution of G for A at position 7256, substitution of C for T at position 7280, and substitution of A for T at position 7280 in the nucleotide sequence of SEQ ID NO. 1, and does not specifically bind to the nucleotide sequences of other nucleic acids.

The primer, probe, or antisense nucleic acid may complementarily bind to the mutated region of the gene, that is, the region including one or more bases selected from the group consisting of positions 4447, 4448, 7255, 7256 and 7280. Further, the primer, probe, or antisense nucleic acid may be used to specifically detect a mutation in one or more bases selected from the group consisting of positions 4447, 4448, 7255, 7256 and 7280 of the gene.

The complementary binding is used herein to mean that antisense nucleic acids are sufficiently complementary to hybridize selectively to a target mTOR mutated gene under the predetermined hybridization or annealing conditions, preferably under physiological conditions, encompassing the terms “substantially complementary” and “perfectly complementary”, preferably perfectly complementary.

The term “antisense nucleic acid” refers to a nucleic acid-based molecule that has a sequence complementary to the target mTOR mutated gene to form a dimer with the mTOR mutated gene, and it can be used for detection of the biomarker mTOR mutated gene of the present invention.

The term “primer” refers to a short nucleic acid sequence having a free 3′ hydroxyl group, ranging in length from 7 to 50 nucleotides, which is able to form base-pairing interaction with a complementary template and serves as a starting point for replication of the template strand. A primer is usually synthesized, but a naturally occurring nucleic acid can be also used. The sequence of the primer does not necessarily have to be exactly identical to that of the template, but must be sufficiently complementary to hybridize with the template. The primer is able to initiate DNA synthesis in the presence of a reagent for polymerization (i.e., DNA polymerase or reverse transcriptase) and four different nucleoside triphosphates at suitable buffers and temperature. In the present invention, epilepsy can be diagnosed by performing PCR amplification using sense and antisense primers of the mTOR nucleotide sequence. PCR conditions and the length of sense and antisense primers can be modified on the basis of the methods known in the art. Preferably, the primer of the present invention may be a primer capable of amplifying the mTOR mutated gene.

The term “probe” refers to a nucleic acid fragment of RNA or DNA capable of specifically binding to mRNA, ranging in length from ones to hundreds of bases. The probe is labeled so as to detect the presence or absence of a specific mRNA. The probe may be prepared in the form of oligonucleotide probe, single stranded DNA probe, double stranded DNA probe, RNA probe or the like. In the present invention, hybridization is performed using a probe complementary to the mTOR mutated gene, and diagnosis can be achieved by the hybridization result. Selection of suitable probe and hybridization conditions can be modified on the basis of the methods known in the art.

In the present invention, the nucleotide sequence of the mTOR mutated gene is revealed, and on the basis of the sequence, those skilled in the art can design the primer or probe capable of specifically amplifying the specific region of the gene.

The primer or probe may be chemically synthesized using a phosphoramidite solid support method or other widely known methods. These nucleic acid sequences may be incorporated with additional features as long as their basic properties are not modified. Examples of the additional features to be incorporated are methylation, capsulation, replacement of one or more native nucleotides with analogues thereof, and inter-nucleotide modifications, but are not limited thereto.

Further, the diagnostic composition for intractable epilepsy caused by malformations of cortical development provided in the present invention may be provided in the form of kit.

The kit of the present invention is able to detect the diagnostic biomarker, mTOR mutated gene or mTOR mutated protein. The kit may include a primer, a probe, or an antisense nucleic acid for the detection of the mTOR mutated gene or the mTOR mutated protein, or optionally, an antibody recognizing the mTOR mutated protein as well as a composition of one or more components, a solution, or an apparatus suitable for the analysis.

In one specific embodiment, the kit for the detection of the mTOR mutated gene of the present invention may be a diagnostic kit for epilepsy, including essential elements required for performing a DNA chip. The DNA chip kit may include a base plate, onto which cDNAs corresponding to the genes or fragments thereof are attached, and reagents, agents and enzymes for preparing fluorescent probes. Also, the base plate may include cDNA corresponding to a quantitative control gene or a fragment thereof. Further, the kit for the detection of the mTOR mutated gene may be a kit including essential elements required for performing PCR. The PCR kit may include test tubes or other suitable containers, reaction buffers (varying in pH and magnesium concentrations), deoxynucleotides (dNTPs), enzymes such as Taq-polymerase, DNase, RNase inhibitor, DEPC water, and sterile water, in addition to a pair of primers specific to the mTOR mutated gene. Further, the kit may include a pair of primers specific to the gene used as a quantitative control.

In another embodiment, the kit for the detection of the mTOR mutated protein of the present invention may include a matrix, a suitable buffer solution, a coloring enzyme, or a secondary antibody labeled with a fluorescent substance, a coloring substrate or the like for the immunological detection of antibody. As for the matrix, a nitrocellulose membrane, a 96-well plate made of polyvinyl resin, a 96-well plate made of polystyrene resin, and a slide glass may be used. As for the coloring enzyme, peroxidase and alkaline phosphatase may be used. As for the fluorescent substance, FITC, RITC or the like may be used, and as for the coloring substrate solution, ABTS (2,2′-azino-bis-(3-ethylbenzthiazoline-6-sulfonic acid)), OPD (o-phenylenediamine), or TMB (tetramethyl benzidine) may be used.

Further, the present invention provides a method for diagnosing intractable epilepsy due to malformations of cortical development, including the steps of treating a sample of a patient with the antibody or aptamer specifically binding to the mTOR mutated protein so as to detect the presence of the protein; and determining that the patient has intractable epilepsy due to malformations of cortical development when the protein is detected in the sample of the patient.

Further, the present invention provides a method for diagnosing intractable epilepsy due to malformations of cortical development, including the steps of treating a sample of a patient with the primer, probe, or antisense nucleic acid complementarily binding to the mTOR mutated gene so as to detect the presence of the gene; and determining that the patient has intractable epilepsy due to malformations of cortical development when the gene is detected in the sample of the patient.

The term “sample of a patient” includes samples such as tissues, cells, etc., in which the mTOR mutated gene or the mTOR mutated protein can be detected. Preferably, it may be a brain tissue sample, but is not limited thereto.

The detection of the mTOR mutated gene in the sample of the patient may be performed by a method including the steps of amplifying the nucleic acid in the sample of the patient using the primer, probe or antisense nucleic acid complementary to the gene, and determining a nucleotide sequence of the amplified nucleic acid.

In detail, the step of amplifying the nucleic acid may be performed by polymerase chain reaction (PCR), multiplex PCR, touchdown PCR, hot start PCR, nested PCR, booster PCR, real-time PCR, differential display PCR (DD-PCR), rapid amplification of cDNA ends (RACE), inverse polymerase chain reaction, vectorette PCR, TAIL-PCR (thermal asymmetric interlaced PCR), ligase chain reaction, repair chain reaction, transcription-mediated amplification, self sustained sequence replication, or selective amplification of the target nucleotide sequence.

Further, the step of determining a nucleotide sequence of the amplified nucleic acid may be performed by Sanger sequencing, Maxam-Gilbert sequencing, Shotgun sequencing, pyrosequencing, hybridization by microarray, allele specific PCR, dynamic allele-specific hybridization (DASH), PCR extension assay, TaqMan technique, automated DNA sequencing, or next-generation DNA sequencing. The next-generation DNA sequencing may be performed using a DNA analyzing system widely known in the art, for example, 454 GS FLX manufactured by Roche, Genome Analyzer manufactured by Illumina, SOLid Platform manufactured by Applied Biosystems, etc.

The detection of the mTOR mutated protein in the sample of the patient may be performed by Western blotting, ELISA, radioimmunoassay, radioimmunodiffusion, ouchterlony immunodiffusion, rocket immunoelectrophoresis, immunohistostaining, immunoprecipitation assay, complement fixation assay, FACS, or protein chip assay using an antibody or aptamer specifically detecting the corresponding amino acid mutation. With the analysis methods, an antigen-antibody complex between the mTOR mutated protein and the antibody thereof can be identified, and intractable epilepsy caused by malformations of cortical development can be diagnosed by examining the antigen-antibody complex between the mTOR mutated protein and the antibody thereof.

The term “antigen-antibody complex” refers to binding products of the mTOR mutated protein and antibodies specific thereto. The formation of the antigen-antibody complex may be determined by measuring the signal intensity of a detection label.

The detection label may be selected from a group consisting of enzymes, fluorescent materials, ligands, luminescent materials, microparticles, Redox molecules, and radioactive isotopes, but not strictly limited thereto. If an enzyme is used as the detect on label, the usable enzyme may include β-glucuronidase, β-D-glycosidase, β-D-galactosidase, urease, peroxidase or alkaline phosphatase, acetylcolinesterase, glucose oxydase, hexokinase and GDPase, RNase, glucose oxydase and luciferase, phosphofructokinase, phosphoenolpyruvate carboxylase, aspartate aminotransferase, phosphenolpyruvate decarboxylase, β-lactamase or the like, but is not limited thereto. The fluorescent material may include fluorecein, isothiocyanate, rhodamine, phycoerythrin, phycocyanin, allophycocyanin, o-phthalaldehyde, fluorescamin or the like, but is not limited thereto. The ligand may include biotin derivatives, but is not limited thereto. The luminescent material may include acridindum ester, luciferin, luciferase or the like, but is not limited thereto. The micro particle may include colloidal gold, colored latex or the like, but is not limited thereto. The Redox molecule may include ferrocene, ruthenium complex, biologen, quinone, Ti ion, Cs ion, diimide, 1,4-benzoquinone, hydroquinone, K₄W(CN)₈, [Os(bpy)₃]²⁺, [RU(bpy)₃]²⁺, [MO(CN)₈]⁴⁻ or the like, but is not limited thereto. The radioactive isotope may include ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, ¹⁸⁶Re or the like, but is not limited thereto.

In one specific embodiment, measurement of the antigen-antibody complex between the mTOR mutated protein and the antibody thereof may be carried out using ELISA assay. The ELISA may include various ELISA assays, including a direct ELISA using a labeled antibody that recognizes antigen attached to a solid support, an indirect ELISA using a labeled antibody that recognizes a capture antibody from the complex of the antibody that recognizes the antigen attached to the solid support, a direct sandwich ELISA using another labeled antibody that recognizes an antigen from the antigen-antibody complex attached to the solid support, or an indirect sandwich ELISA which reacts the antigen-antibody complex attached to the solid support with another antibody that recognizes an antigen and then uses labeled secondary antibody that recognizes the another antibody. More preferably, a sandwich ELISA assay may be used, in which an antibody is attached to a solid support and reacted with a sample, followed by attachment of a labeled antibody that recognizes antigen of the antigen-antibody complex for enzymatic staining, or attachment of labeled secondary antibody with respect to the antibody that perceives the antigen of the antigen-antibody complex for enzymatic staining. Development of intractable epilepsy caused by malformations of cortical development can be examined by identifying the complex formation between the diagnostic biomarker mTOR mutated protein and antibody.

In another embodiment, Western blot may be carried out using one or more antibodies against the mTOR mutated protein. For example, the entire protein is isolated from the sample, and through electrophoresis, the proteins are divided according to sizes thereof. The proteins are then transferred onto a nitrocellulose membrane and reacted with the antibody. By checking the amount of generated antigen-antibody complex using the labeled antibodies, it is possible to determine whether epilepsy is developed, based on the amount of the mTOR mutated protein generated due to expression of the mTOR mutated gene. Such detection may be carried out by investigating the antigen-antibody complex between the mTOR mutated protein and the antibody thereof.

Further, in still another embodiment, a protein chip may be used, in which one or more antibodies against the mTOR mutated protein are arranged on a predetermined location of a substrate and immobilized in high density. The method of analyzing a sample using the protein chip may include isolating the protein from the sample, hybridizing the isolated protein with the protein chip to form an antigen-antibody complex, reading the result to identify the presence of the protein, and determining whether intractable epilepsy caused by malformations of cortical development is developed.

Intractable epilepsy caused by malformations of cortical development can be diagnosed, when the mTOR mutated gene or the mTOR mutated protein is detected by the above detection methods.

Further, the present invention provides a composition for inducing intractable epilepsy due to malformations of cortical development, including the mTOR mutated protein.

Further, the present invention provides a composition for inducing intractable epilepsy due to malformations of cortical development, including the mTOR mutated gene.

Further, the present invention provides a method for inducing intractable epilepsy due to malformations of cortical development, including the step of introducing the protein into a cell or an individual.

Further, the present invention provides a method for inducing intractable epilepsy due to malformations of cortical development, including the step of introducing the gene into a cell or an individual.

As used herein, the term “induction” means induction of a change from a normal state into a pathological state. With respect to the objects of the present invention, the induction means that epilepsy is developed from the normal state. Preferably, epilepsy may be intractable epilepsy caused by malformations of cortical development.

In one embodiment, epilepsy-induced cells can be prepared by introducing the mTOR mutated gene or the mTOR mutated protein into cells. The cells include brain cells or embryos. When the mTOR mutated gene or the mTOR mutated protein is introduced, excessive mTOR activation occurs by mTOR mutations to generate neuronal migration disorders and to dramatically increase S6 protein phosphorylation, leading to epilepsy.

The mTOR protein or the mTOR protein having mutations in the amino acid sequence can be obtained from the natural source by extraction and purification using a method widely known in the art. Otherwise, the mTOR protein having mutations in the amino acid sequence can be chemically synthesized (Merrifleld, J. Amer. Chem. Soc. 85:2149-2156, 1963) or can be obtained by a recombinant DNA technology.

When the protein is chemically synthesized, it can be obtained by a polypeptide synthetic method widely known in the art. When the recombinant DNA technology is used, a nucleic acid encoding the mTOR protein having mutations in the amino acid sequence is inserted into a suitable expression vector, a host cell is transformed with the vector and then cultured to express the mTOR protein having mutations in the amino acid sequence, and the mTOR protein having mutations in the amino acid sequence is recovered from the host cell. The protein is expressed in the selected host cell, and then a typical biochemical separation technique, for example, treatment by use of a protein precipitant (salting-out), centrifugation, sonication, ultrafiltration, dialysis, a variety of chromatographies such as molecular sieve chromatography (gel filtration), adsorption chromatography, ion-exchange chromatography, or affinity chromatography can be used for separation and purification. Typically, in order to separate a highly pure protein, combinations thereof are used.

The nucleotide sequence encoding the mTOR protein or the mTOR protein having mutations in the amino acid sequence can be isolated from the natural source or prepared by a chemical synthetic method. The nucleic acid having the nucleotide sequence may be single- or double-stranded, and it may be a DNA molecule (genome, cDNA) or an RNA molecule. When the nucleic acid is chemically synthesized, a synthetic method widely known in the art, for example, a method described in the literature (Engels and Uhlmann, Angew Chem Int Ed Engl. 37:73-127, 1988) may be used, and examples thereof may include triester, phosphite, phosphoramidite and H-phosphonate methods, PCR and other autoprimer methods, oligonucleotide synthesis on solid supports or the like.

The mTOR mutated protein or mutated gene of the present invention may be introduced into cells, and preferably, brain cells. In addition, it may be introduced into embryos, and preferably, embryos at the stage of brain formation and development.

The introduction method of the protein or the gene is not particularly limited. For example, a vector may be introduced into cells via a method such as transformation, transfection or transduction. The vector introduced into cells continuously expresses the gene in the cells so as to produce the mTOR protein having mutations in the amino acid sequence.

The present invention provides a technique for diagnosing epilepsy using the mTOR gene having mutations in the nucleotide sequence or the mTOR protein having mutations in the amino acid sequence, and in particular, it is effective for the diagnosis of patients with intractable epilepsy caused by malformations of cortical development. Further, the present invention provides a technique for inducing epilepsy using the mTOR gene having mutations in the nucleotide sequence or the mTOR protein having mutations in the amino acid sequence. Accordingly, it is possible to conduct studies on gene functions and molecular mechanisms of epilepsy, and exploration of novel anti-epileptic drugs using the epilepsy animal model thus prepared.

One or more embodiments of the present invention will now be described in further detail with reference to the following Examples. However, these examples are for the illustrative purposes only and are not intended to limit the scope of the invention.

### EXAMPLE 1

### Identification of Brain Somatic Mutations I

1.1. Sample of Epilepsy Patient

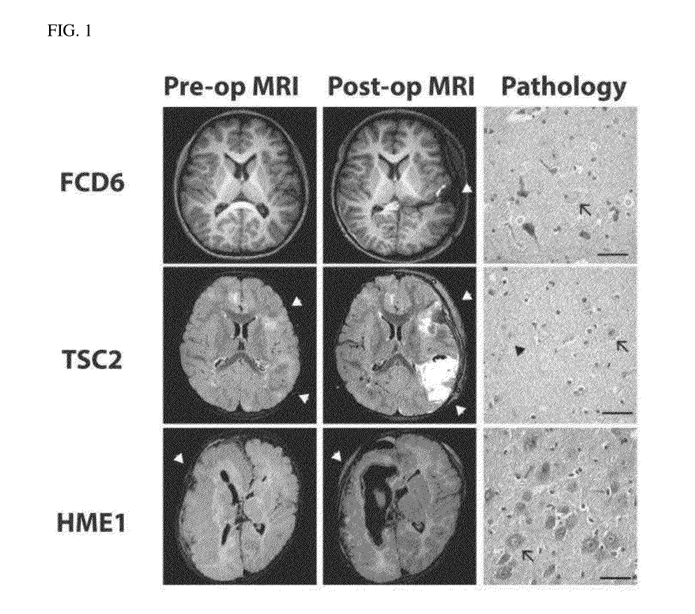

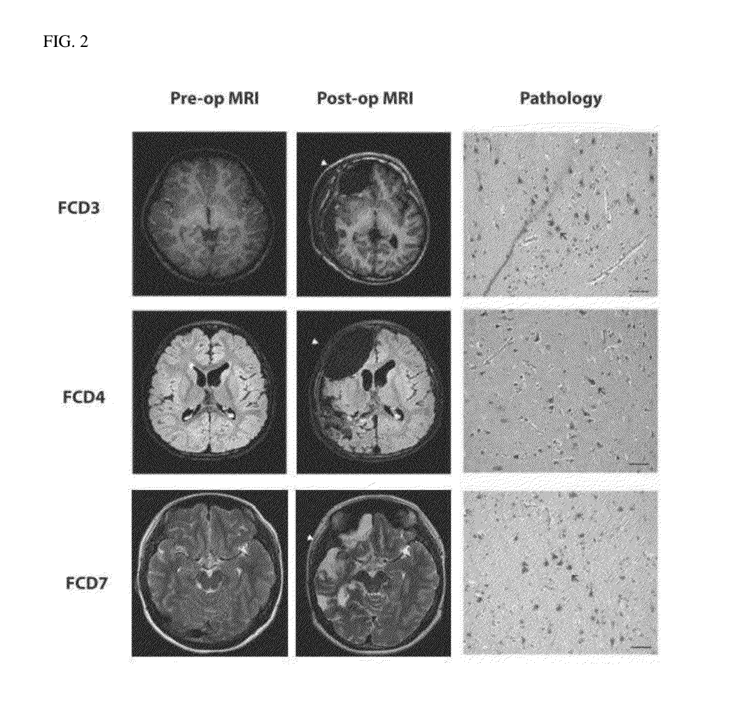

Blood (about 5 ml) and brain tissue (about 1-2 g) were obtained with consent from 6 patients after surgery for intractable epilepsy due to malformations of cortical development (Pediatric Neurosurgery, Severance Hospital). 6 patients with malformations of cortical development were composed of 4 patients with focal cortical dysplasia (FCD), 1 patient with hemimegalencephaly (HME) and 1 patient with Tuberous sclerosis complex (TSC) 1 (FIG. 1 and FIG. 2).

1.2. Whole Exome Sequencing

Genomic DNAs were isolated from the blood and brain tissue samples of 6 patients using a Qiamp mini kit (Qiagen). Then, exome enrichment was carried out using a sure select target enrichment system (Agilent). For more accurate analysis of gene mutations in genomic DNAs of the blood and brain tissue samples of the patients using Hiseq2000 (Illumina), whole exome sequencing with ˜500× coverage on average, which is 5 times higher than the general coverage, was performed.

1.3. Analysis of Gene Mutations Specific to Encephalopathy

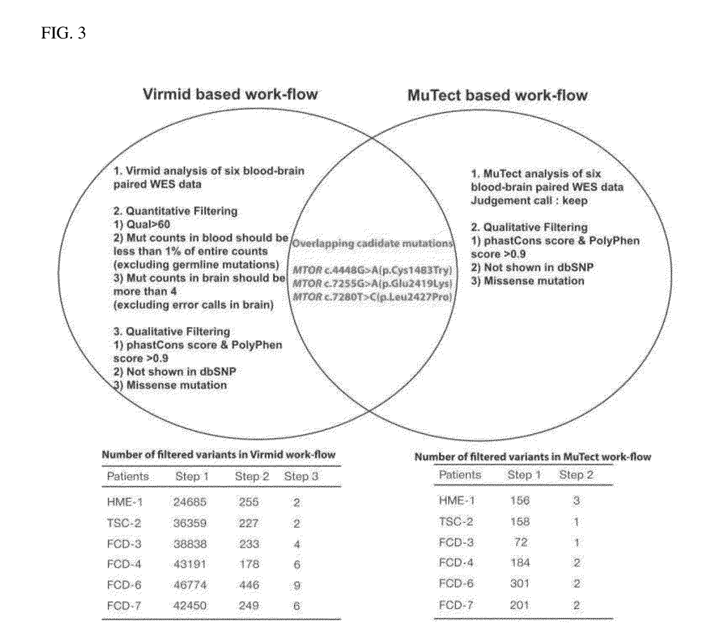

About 70 GB of exome sequencing information per 1 patient was obtained from the results of Example 1.2. As a bioinformatics tool for analysis of gene mutations specific to encephalopathy, algorithms using Virmid (Genome Biology, 14 (8), R90, 2013) and MuTect software (Nature Biotechnology, 31, 213-219 (2013)) at the same time were developed. Therefore, a common causative gene and genetic mutations that are specifically present in encephalopathy were found in 6 patients (FIG. 3).

1.4. Identification of Genetic Mutations in Epilepsy Patients

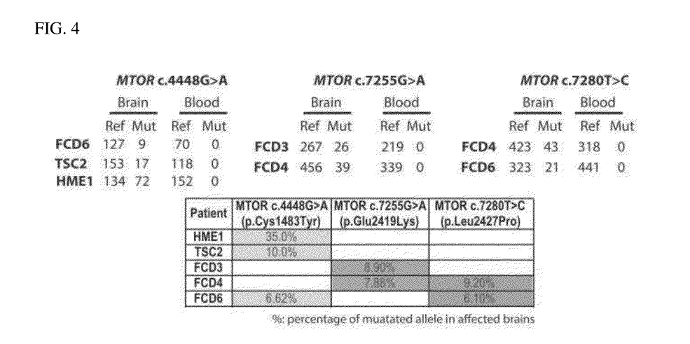

The results of Example 1.3 showed that presence of 3 types of common genetic mutations in the mTOR gene was found in 5 patients out of 6 patients with intractable epilepsy caused by malformations of cortical development. Such mTOR gene mutations were not found in the blood, but in the brain tissues, and the rate of mutated allele in affected regions of brain was as low as about 6% (FIG. 4).

In detail, the genetic mutations were found to be substitution of A for G at position 4448, substitution of A for G at position 7255, and substitution of C for T at position 7280 in the nucleotide sequence of SEQ ID NO. 1 of the mTOR gene. Such genetic mutations were found to lead to substitution of Y for C at position 1483, substitution of K for E at position 2419, and substitution of P for L at position 2427 in the amino acid sequence of SEQ ID NO. 2 of the mTOR protein.

Further, it was found that 3 patients have a substitution of A for G at position 4448 in the nucleotide sequence of SEQ ID NO. 1 of the mTOR gene, 2 patients have a substitution of A for G at position 7255 in the nucleotide sequence of SEQ ID NO. 1, 2 patients have a substitution of C for T at position 7280 in the nucleotide sequence of SEQ ID NO. 1, and 2 patients have one or more mutations of the three genetic substitutions, indicating that epilepsy can be caused by two or more genetic mutations as well as one genetic mutation.

Further, the mutations in the nucleotide sequence of the mTOR gene resulted in mutations in the amino acid sequence of the mTOR protein, in which 3 patients have a substitution of Y for C at position 1483 in the amino acid sequence of SEQ ID NO. 2 of the mTOR protein, 2 patients have a substitution of K for E at position 2419 in the amino acid sequence of SEQ ID NO. 2, 2 patients have a substitution of P for L at position 2427 in the amino acid sequence of SEQ ID NO. 2, and 2 patients have one or more mutations of the three amino acid mutations, indicating that epilepsy can be caused by two or more amino acid mutations as well as one amino acid mutation.

### EXAMPLE 2

### Identification of Brain Somatic Mutations II

2.1. Sample of Epilepsy Patient

Saliva (about 1 ml) and formalin-fixed, paraffin-embedded brain tissue were obtained with consent from 76 patients after surgery for intractable epilepsy due to malformations of cortical development (Pediatric Neurosurgery and Pediatric Neurology, Severance Hospital). Of 76 patients, 51 patients were diagnosed with focal cortical dysplasia type IIa (FCDIIa) and 25 patients were diagnosed with focal cortical dysplasia type IIb (FCDIIb).

2.2. Targeted Re-Sequencing

Genomic DNAs were isolated from the saliva and formalin-fixed, paraffin-embedded brain tissue samples of 76 patients prepared in Example 2.1 using a Qiamp mini DNA kit (Qiagen) and a prepIT-L2P purification kit (DNAgenotek). Then, two pairs of primers having two targets were prepared so that they contained the mTOR targeted codon region (containing amino acids, Cys1483, Glu2419, and Leu2427).

TABLE 2

| | | | SEQ |
|---|---|---|---|
| Target | | | ID |
| region | primer | | NO. |
| Chr1: | forward | 5′-TAGGTTACAGGCCTGGATGG-3′ | 3 |
| 11174301 | | | |
| ~Chr1: | reverse | 5′-CTTGGCCTCCCAAAATGTTA-3′ | 4 |
| 11174513 | | | |
| Chr1: | forward | 5′-TCCAGGCTACCTGGTATGAGA-3′ | 5 |
| 11217133 | | | |
| ~Chr1: | reverse | 5′-GCCTTCCTTTCAAATCCAAA-3′ | 6 |
| 11217344 | | | |

Each primer contains a patient-specific index, and one index per one sample of a patient was used. Therefore, the origin of the nucleotide sequence can be determined during analysis of the genetic mutations. PCR of the target site was performed using the primers thus prepared so as to amplify two targeted nucleotide sequences. Then, a DNA library was prepared using a Truseq DNA kit (Illumina) and targeted re-sequencing was performed using a Miseq or Hiseq sequencer (Illumina).

2.3. Identification of Gene Mutations Present in Specific Region of Target Gene

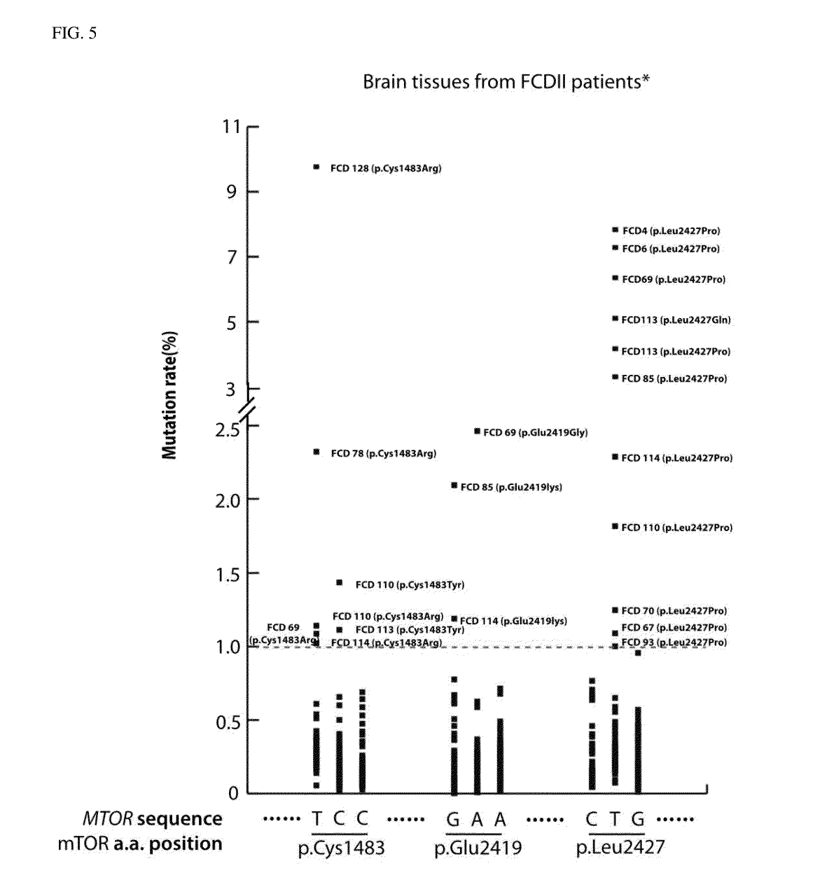

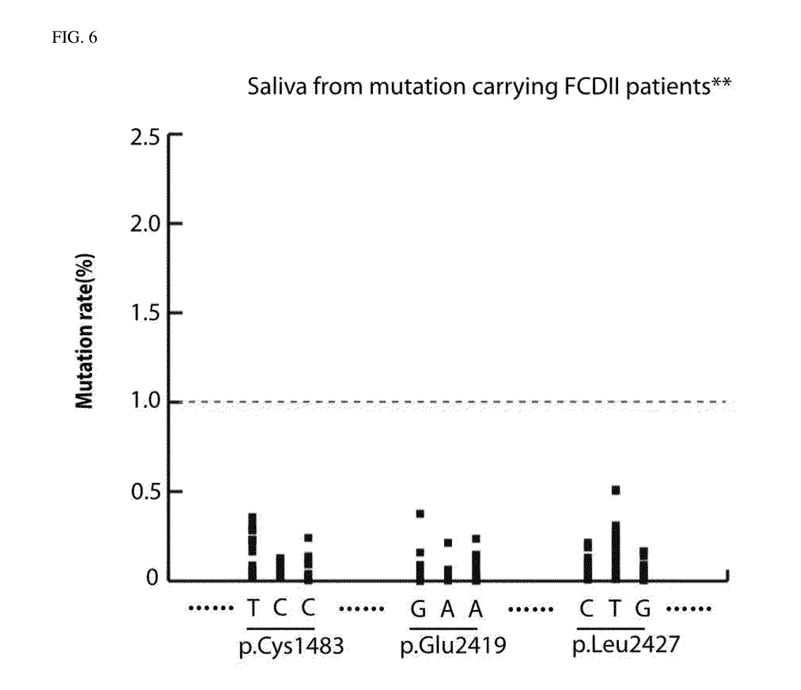

Sequencing information of the target region with 1156˜348630× coverage per 1 patient was obtained from the results of Example 2.2. As a tool for analysis of genetic mutations, IGV viewer (www.broadinstitute.org/igv/home) and in-house python script were used. When the genetic mutation rate was higher than 1%, it was determined as a genetic mutation. FIG. 5 and FIG. 6 illustrate the genetic mutation rates of the target region in the formalin-fixed, paraffin-embedded brain tissue and saliva.

2.4 Identification of Genetic Mutations in Epilepsy Patients

The results of Example 2.3 showed that 6 types of genetic mutations in the target region of the mTOR gene were found in 10 patients, and 3 types of them are genetic mutations newly identified by targeted re-sequencing (Table 3).

TABLE 3

| Patients/ | Age at | | | Nucleotide | Protein | % Mutated |
|---|---|---|---|---|---|---|
| Sex | Surgery | Pathology | MRI report | changes | changes | allele |
| FCD67/M | 8 yr | Cortical dyslamination, | Encephalomalacia | 4447T>C | 1483C>R | 1.21 |
| | 10 m | Dysmorphic neurons, | involving right | 7280T>C | 2427L>P | 1.09~3.98 |
| | | consistent with FCDIIa | parietooccipital lobe | | | |
| FCD69/F | 3 yr | Cortical dyslamination, | Diffuse cortical dysplasia | 4447T>C | 1483C>R | 1.03 |
| | 5 m | Dysmorphic neurons, | in the Rt. Frontal lobe | 7256A>G | 2419E>G | 2.46 |
| | | consistent with FCDIIa | | 7280T>C | 2427L>P | 1.79~6.35 |
| FCD70/F | l yr | Cortical dyslamination, | Cortical dysplasia in left | 7280T>C | 2427L>P | 1.25~3.86 |
| | 8 m | Dysmorphic neurons, | insular area, frontal lobe | | | |
| | | consistent with FCDIIa | side, right frontal lobe | | | |
| | | | area | | | |
| FCD78/M | 12 yr | Cortical dyslamination, | Dysplastic cortex, | 4447T>C | 1483C>R | 2.05~2.41 |
| | 1 m | Dysmorphic neurons, | Lt. temporal pole | | | |
| | | consistent with FCDIIa | | | | |
| FCD85/F | 17 yr | Cortical dyslamination, | No abnormal signal | 7255G>A | 2419E>K | 2.09 |
| | 11 m | Dysmorphic neurons, | intensity | 7280T>C | 2427L>P | 3.31~4.07 |
| | | consistent with FCDIIa | | | | |
| FCD93/F | 3 yr | Cortical dyslamination, | Cortical dysplasia | 7280T>C | 2427L>P | 1.00~1.86 |
| | 10 m | Dysmorphic neurons, | involving right | | | |
| | | consistent with FCDIIa | frontoparietal lobe and | | | |
| | | | right posterior temporal | | | |
| | | | lobe | | | |
| FCD110/F | 14 yr | Cortical dyslamination, | No abnormal signal | 4447T>C | 1483C>R | 1.09~1.14 |
| | 1 m | Dysmorphic neurons, | intensity | 4448G>A | 1483C>Y | 1.44 |
| | | balloon cells, | | 7280T>C | 2427L>P | 1.81~4.30 |
| | | consistent with FCDIIb | | | | |
| FCD113/F | 10 yr | Cortical dyslamination, | Cortical dysplasia | 4448G>A | 1483C>Y | 1.11 |
| | | Dysmorphic neurons, | involving left temporal | 7280T>A | 2427L>Q | 2.86~5.11 |
| | | balloon cells, | lobe and occipital lobe | 7280T>C | 2427L>P | 4.17 |
| | | consistent with FCDIIb | | | | |
| FCD114/M | 7 yr | Cortical dyslamination, | Cortical dysplasia, | 4447T>C | 1483C>R | 1.02 |
| | 10 m | Dysmorphic neurons, | left middle frontal gyrus | 7255G>A | 2419E>K | 1.18 |
| | | balloon cells, | | 7280T>C | 2427L>P | 2.29~3.88 |
| | | consistent with FCDIIb | | | | |
| FCD128/F | 4 yr | Cortical dyslamination, | Cortical dysplasia, | 4447T>C | 1483C>R | 6.61~9.77 |
| | 4 m | Dysmorphic neurons, | right frontal gyrus | | | |
| | | balloon cells, | | | | |
| | | consistent with FCDIIb | | | | |

Such mTOR gene mutations were not found in the saliva, but in the formalin-fixed, paraffin-embedded brain tissues (FIG. 5 and FIG. 6). It was also found that the genetic mutation rate ranges from 1.03% to 9.77%.

The genetic mutations newly identified were found to be substitution of C for T at position 4447, substitution of G for A at position 7256, and substitution of A for T at position 7280 in the nucleotide sequence of SEQ ID NO. 1 of the mTOR gene (nucleotide sequence of wild-type mTOR gene). Such genetic mutations were found to lead to substitution of R for C at position 1483, substitution of G for E at position 2419, and substitution of Q for L at position 2427 in the amino acid sequence of SEQ ID NO. 2 of the mTOR protein (amino acid sequence of wild-type mTOR protein).

Further, it was found that 6 patients have a substitution of C for T at position 4447 in the nucleotide sequence of SEQ ID NO. 1 of the mTOR gene, 1 patient has a substitution of G for A at position 7256 in the nucleotide sequence of SEQ ID NO. 1, 1 patient has a substitution of A for T at position 7280 in the nucleotide sequence of SEQ ID NO. 1, and 6 patients have one or more mutations of the three genetic substitution mutations, indicating that epilepsy can be caused by one or more genetic mutations.

Further, the mutations in the nucleotide sequence of the mTOR gene resulted in mutations in the amino acid sequence of the mTOR protein, in which 6 patients have a substitution of R for C at position 1483 in the amino acid sequence of SEQ ID NO. 2 of the mTOR protein, 1 patient has a substitution of G for E at position 2419 in the amino acid sequence of SEQ ID NO. 2, 1 patient has a substitution of Q for L at position 2419 in the amino acid sequence of SEQ ID NO. 2, and 6 patients have one or more mutations of the three amino acid substitution mutations, indicating that epilepsy can be caused by one or more amino acid mutations.

### EXAMPLE 3

### Induction of Intractable Epilepsy Using mTOR Mutated Gene

3.1 Induction of mTOR Mutation and Preparation of mTOR Mutant Construct

pcDNA3.1 flag-tagged wild-type mTOR construct was provided by Dr. Kun-Liang Guan at the University of California, San Diego. The construct was used together with a QuikChange II site-directed mutagenesis kit (200523, Stratagene, USA) to prepare mTOR mutant vectors (C1483R, E2419G, L2427Q, C1483Y, E2419K and L2427P).

To prepare a pCIG-mTOR mutant-IRES-EGFP vector, MfeI and MluI restriction enzyme sites were first inserted into pCIG2 (CAG promoter-MCS-IRES-EGFP) using the following annealing primers [forward primer 5′-AATTCCAATTGCCCGGGCTTAAGATCGATACGCGTA-3′ (SEQ ID NO. 19) and reverse primer 5′-ccggtacgcgtatcgatcttaagcccgggcaattgg-3′ (SEQ ID NO. 20)) so as to prepare pCIG-C1. Subcloning of the newly inserted MfeI and MluI restriction enzyme sites was carried out using the following primers [hmTOR-MfeI-flag-F; gATcACAATTGTGGCCACCATGGACTACAAGGACGACGATGACA AGatgc (SEQ ID NO. 21), and hmTOR-MluI-R; tgatcaACGCGTttaccagaaagggcaccagccaatatagc (SEQ ID NO. 22)] so as to prepare pCIG-mTOR wild type-IRES-EGFP and pCIG-mTOR mutant-IRES-EGFP vectors. Table 4 indicates primers used for inducing mutation.

TABLE 4

| | | | SEQ ID |
|---|---|---|---|
| | primer | | NO. |
| C1483R | forward | 5′-GGCCTCGAGGCGGCGCATGCGGC-3′ | 7 |
| | reverse | 5′-GCCGCATGCGCCGCCTCGAGGCC-3′ | 8 |
| E2419G | forward | 5′-GTCATGGCCGTGCTGGGAGCCTTTGTCTATGAC-3′ | 9 |
| | reverse | 5′-GTCATAGACAAAGGCTCCCAGCACGGCCATGAC-3′ | 10 |
| L2427Q | forward | 5′-GTCTATGACCCCTTGCAGAACTGGAGGCTGATG-3′ | 11 |
| | reverse | 5′-CATCAGCCTCCAGTTCTGCAAGGGGTCATAGAC-3′ | 12 |
| C1483Y | forward | GCCGCATGCGCTACCTCGAGGCC | 13 |
| | reverse | GGCCTCGAGGTAGCGCATGCGGC | 14 |
| E2419K | forward | GTGTCATGGCCGTGCTGAAAGCCTTTGTCTATGAC | 15 |
| | reverse | GTCATAGACAAAGGCTTTCAGCACGGCCATGACAC | 16 |
| L2427P | forward | GTCTATGACCCCTTGCCGAACTGGAGGCTGATG | 17 |
| | reverse | CATCAGCCTCCAGTTCGGCAAGGGGTCATAGAC | 18 |

3.2. Cell Culture, Transfection, and Western Blot

HEK293T cells (thermoscientific) were cultured in DMEM (Dulbecco's Modified Eagle's Medium) containing 10% FBS under the conditions of 37° C. and 5% CO₂. The cells were transfected with empty flag-tagged vector, flag-tagged wild-type mTOR and flag-tagged mutant mTOR using a jetPRIME transfection reagent (Polyplus, France). For 24 hours after transfection, the cells were serum-starved in DMEM containing 0.1% FBS, and cultured in PBS containing 1 mM MgCl₂ and CaCl₂ under the conditions of 37° C. and 5% CO₂ for 1 hour. The cells were lysed in PBS containing 1% Triton X-100, Halt protease, and phosphatase inhibitor cocktail (78440, Thermo Scientific, USA). Proteins were resolved on SDS-PAGE and transferred to a PVDF membrane (Milipore, USA). The membrane was blocked with 3% BSA in TBS containing 0.1% Tween 20 (TBST). Thereafter, the membrane was washed with TBST four times, repeatedly. The membrane was incubated with a 1:1000 dilution of primary antibodies containing anti-phospho-S6-ribosomal protein (5364, Cell Signaling Technology, USA), anti-S6 ribosomal protein (2217, Cell Signaling Technology, USA) and anti-flag M2 (8164, Cell Signaling Technology, USA) in TBST at 4° C. overnight. After incubation, the membrane was washed with TBST four times, repeatedly. Then, the membrane was incubated with a 1/5000 dilution of HRP-linked anti-rabbit or anti-mouse secondary antibodies (7074, Cell Signaling Technology, USA) at room temperature for 2 hours. The membrane was washed with TBST, and immunodetection was performed using an ECL reaction.

The transfected mTOR mutants were a flag-tagged mTOR mutant expressing a protein having a substitution of arginine (R) for cysteine (C) at position 1483 in the amino acid sequence of SEQ ID NO. 2, a flag-tagged mTOR mutant expressing a protein having a substitution of glycine (G) for glutamic acid (E) at position 2419 in the amino acid sequence of SEQ ID NO. 2, and a flag-tagged mTOR mutant expressing a protein having a substitution of glutamine (Q) for leucine (L) at position 2427 in the amino acid sequence of SEQ ID NO. 2. Further, the transfected mTOR mutants were a flag-tagged mTOR mutant expressing a protein having a substitution of tyrosine (Y) for cysteine (C) at position 1483 in the amino acid sequence of SEQ ID NO. 2, a flag-tagged mTOR mutant expressing a protein having a substitution of lysine (K) for glutamic acid (E) at position 2419 in the amino acid sequence of SEQ ID NO. 2, and a flag-tagged mTOR mutant expressing a protein having a substitution of proline (P) for leucine (L) at position 2427 in the amino acid sequence of SEQ ID NO. 2.

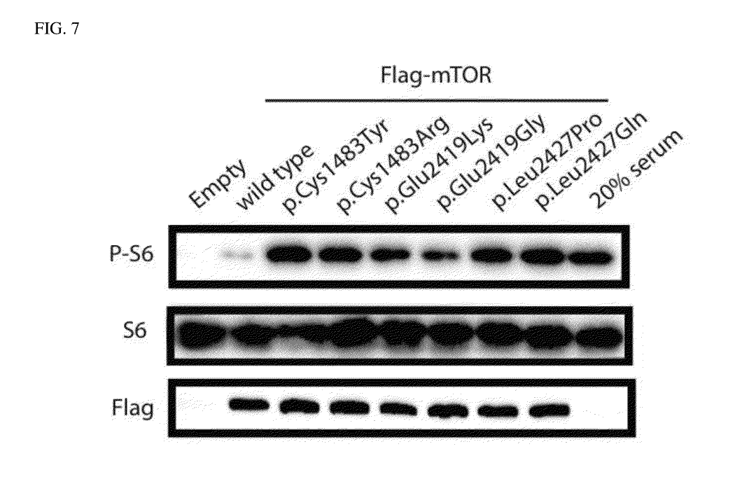

As a result, when the mTOR mutants were transfected, mTOR hyperactivation was observed. The hyperactivation was caused by the mTOR mutants, which was confirmed by phosphorylated S6 protein as an indicator of mTOR activation (FIG. 7).

3.3. In Vitro mTOR Kinase Assay

Phosphorylation activity of mTOR was measured using a K-LISA mTOR activity kit (CBA055, Calbiochem, USA) in accordance with the manufacturer's protocol. The transfected cells (HEK293T cell) were lysed in TBS containing 1% Tween 20, Halt protease and phosphatase inhibitor cocktail. 1 mg of the whole lysate was pre-cleared by adding 15 ul of protein G-beads (10004D, Life technologies, USA) and incubated at 4° C. for 15 minutes. Anti-flag antibody was added to the pre-cleared lysate and incubated at 4° C. overnight. 50 ul of 20% slurry of protein G-beads were added and incubated at 4° C. for 90 minutes. The supernatant was carefully discarded. The pelleted beads were washed with 500 ul of lysis buffer four times, repeatedly and washed once with 1× kinase buffer which was contained in the K-LISA mTOR activity kit. The pelleted beads were re-suspended with 50 ul of 2× kinase buffer and 50 ul of mTOR substrate (p70S6K-GST fusion protein) and incubated at 30° C. for 30 minutes. The reaction mixture was incubated in a Glutathione-coated 96-well plate at 30° C. for 30 minutes. Anti-p70S6K-pT389 antibody, HRP antibody-conjugate and TMB substrate were used to detect the phosphorylated substrate. The relative activity was determined by measuring absorbance at 450 nm.

The transfected cells were cells that were transfected with the flag-tagged mTOR mutant expressing a protein having a substitution of arginine (R) for cysteine (C) at position 1483 in the amino acid sequence of SEQ ID NO. 2, the flag-tagged mTOR mutant expressing a protein having a substitution of glycine (G) for glutamic acid (E) at position 2419 in the amino acid sequence of SEQ ID NO. 2, and the flag-tagged mTOR mutant expressing a protein having a substitution of glutamine (Q) for leucine (L) at position 2427 in the amino acid sequence of SEQ ID NO. 2. Further, the transfected cells were cells that were transfected with the flag-tagged mTOR mutant expressing a protein having a substitution of tyrosine (Y) for cysteine (C) at position 1483 in the amino acid sequence of SEQ ID NO. 2, the flag-tagged mTOR mutant expressing a protein having a substitution of lysine (K) for glutamic acid (E) at position 2419 in the amino acid sequence of SEQ ID NO. 2, and the flag-tagged mTOR mutant expressing a protein having a substitution of proline (P) for leucine (L) at position 2427 in the amino acid sequence of SEQ ID NO. 2.

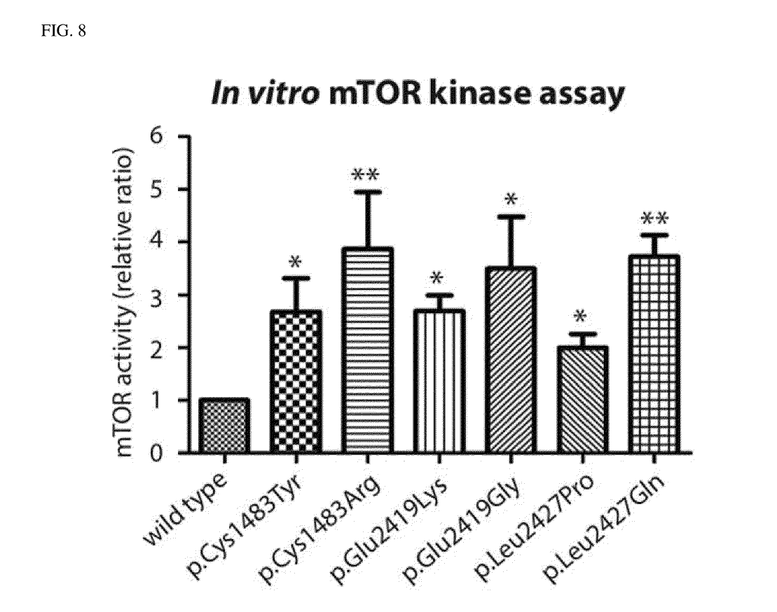

As a result, greatly increased mTOR kinase activity due to six types of the mutants was observed in the cells transfected with the mTOR mutants (FIG. 8), indicating that epilepsy can be caused by the mTOR gene or protein having such mutations.

What is claimed is:

1. An isolated protein consisting of an amino acid sequence which comprises one or more mutations selected from the group consisting of
substitution of tyrosine (Y) for cysteine (C) at position 1483,
substitution of arginine (R) for cysteine (C) at position 1483,
substitution of lysine (K) for glutamic acid (E) at position 2419,
substitution of glycine (G) for glutamic acid (E) at position 2419,
substitution of proline (P) for leucine (L) at position 2427, and
substitution of glutamine (Q) for leucine (L) at position 2427
in an amino acid sequence of SEQ ID NO. 2.

2. The isolated protein of claim 1, wherein the protein having the substitution of tyrosine (Y) for cysteine (C) at position 1483 is encoded by a gene having substitution of adenine (A) for guanine (G) at position 4448 in a nucleotide sequence of SEQ ID NO. 1,
the protein having the substitution of arginine (R) for cysteine (C) at position 1483 is encoded by a gene having substitution of cytosine (C) for thymine (T) at position 4447 in the nucleotide sequence of SEQ ID NO. 1,
the protein having the substitution of lysine (K) for glutamic acid (E) at position 2419 is encoded by a gene having substitution of adenine (A) for guanine (G) at position 7255 in the nucleotide sequence of SEQ ID NO. 1,
the protein having the substitution of glycine (G) for glutamic acid (E) at position 2419 is encoded by a gene having substitution of guanine (G) for adenine (A) at position 7256 in the nucleotide sequence of SEQ ID NO. 1,
the protein having the substitution of proline (P) for leucine (L) at position 2427 is encoded by a gene having substitution of cytosine (C) for thymine (T) at position 7280 in the nucleotide sequence of SEQ ID NO. 1, and
the protein having the substitution of glutamine (Q) for leucine (L) at position 2427 is encoded by a gene having substitution of adenine (A) for thymine (T) at position 7280 in the nucleotide sequence of SEQ ID NO. 1.

3. An isolated gene consisting of a nucleotide sequence which comprises one or more mutations selected from the group consisting of
substitution of adenine (A) for guanine (G) at position 4448,
substitution of cytosine (C) for thymine (T) at position 4447,
substitution of adenine (A) for guanine (G) at position 7255,
substitution of guanine (G) for adenine (A) at position 7256,
substitution of cytosine (C) for thymine (T) at position 7280, and
substitution of adenine (A) for thymine (T) at position 7280
in a nucleotide sequence of SEQ ID NO. 1.

4. A composition comprising the protein of claim 1; or an antibody or aptamer specifically binding to the protein of claim 1.

5. The composition of claim 4, wherein the antibody or aptamer specifically binds to a region including a mutation in one or more amino acid residues selected from the group consisting of positions 1483, 2419 and 2427 of the protein.

6. A method for diagnosing intractable epilepsy due to malformations of cortical development, comprising the steps of:
treating a sample of a patient with an antibody or aptamer specifically binding to the protein of claim 1 so as to detect the presence of the protein of claim 1; and
determining that the patient has intractable epilepsy due to malformations of cortical development when the protein of claim 1 is detected in the sample of the patient.

7. The method of claim 6, wherein the antibody or aptamer is able to specifically detect a mutation in one or more amino acid residues selected from the group consisting of positions 1483, 2419 and 2427 of the protein.

8. A composition comprising the gene of claim 3; or a primer, probe, or antisense nucleic acid complementarily binding to the gene of claim 3.

9. The composition of claim 8, wherein the primer, probe, or antisense nucleic acid complementarily binds to a region including one or more bases selected from the group consisting of positions 4447, 4448, 7255, 7256 and 7280 of the gene.

10. A method for diagnosing intractable epilepsy due to malformations of cortical development, comprising the steps of:
treating a sample of a patient with a primer, probe, or antisense nucleic acid complementarily binding to the gene of claim 3 so as to detect the presence of the gene of claim 3; and
determining that the patient has intractable epilepsy due to malformations of cortical development when the gene of claim 3 is detected in the sample of the patient.

11. The method of claim 10, wherein the primer, probe, or antisense nucleic acid is able to specifically detect a mutation in one or more bases selected from the group consisting of positions 4447, 4448, 7255, 7256 and 7280 of the gene.


SEQUENCE LISTING (22 sequences)

SEQ ID NO: 1 - DNA, 7650 bp, Homo sapiens
  gene (1)..(7650): wild type mTOR
atgcttggaaccggacctgccgccgccaccaccgctgccaccacatctagcaatgtgagcgtcctgcagcagtttgccagtggcctaaagagccggaatgaggaaaccagggccaaagccgccaaggagctccagcactatgtcaccatggaactccgagagatgagtcaagaggagtctactcgcttctatgaccaactgaaccatcacatttttgaattggtttccagctcagatgccaatgagaggaaaggtggcatcttggccatagctagcctcataggagtggaaggtgggaatgccacccgaattggcagatttgccaactatcttcggaacctcctcccctccaatgacccagttgtcatggaaatggcatccaaggccattggccgtcttgccatggcaggggacacttttaccgctgagtacgtggaatttgaggtgaagcgagccctggaatggctgggtgctgaccgcaatgagggccggagacatgcagctgtcctggttctccgtgagctggccatcagcgtccctaccttcttcttccagcaagtgcaacccttctttgacaacatttttgtggccgtgtgggaccccaaacaggccatccgtgagggagctgtagccgcccttcgtgcctgtctgattctcacaacccagcgtgagccgaaggagatgcagaagcctcagtggtacaggcacacatttgaagaagcagagaagggatttgatgagaccttggccaaagagaagggcatgaatcgggatgatcggatccatggagccttgttgatccttaacgagctggtccgaatcagcagcatggagggagagcgtctgagagaagaaatggaagaaatcacacagcagcagctggtacacgacaagtactgcaaagatctcatgggcttcggaacaaaacctcgtcacattacccccttcaccagtttccaggctgtacagccccagcagtcaaatgccttggtggggctgctggggtacagctctcaccaaggcctcatgggatttgggacctcccccagtccagctaagtccaccctggtggagagccggtgttgcagagacttgatggaggagaaatttgatcaggtgtgccagtgggtgctgaaatgcaggaatagcaagaactcgctgatccaaatgacaatccttaatttgttgccccgcttggctgcattccgaccttctgccttcacagatacccagtatctccaagataccatgaaccatgtcctaagctgtgtcaagaaggagaaggaacgtacagcggccttccaagccctggggctactttctgtggctgtgaggtctgagtttaaggtctatttgcctcgcgtgctggacatcatccgagcggccctgcccccaaaggacttcgcccataagaggcagaaggcaatgcaggtggatgccacagtcttcacttgcatcagcatgctggctcgagcaatggggccaggcatccagcaggatatcaaggagctgctggagcccatgctggcagtgggactaagccctgccctcactgcagtgctctacgacctgagccgtcagattccacagctaaagaaggacattcaagatgggctactgaaaatgctgtccctggtccttatgcacaaaccccttcgccacccaggcatgcccaagggcctggcccatcagctggcctctcctggcctcacgaccctccctgaggccagcgatgtgggcagcatcactcttgccctccgaacgcttggcagctttgaatttgaaggccactctctgacccaatttgttcgccactgtgcggatcatttcctgaacagtgagcacaaggagatccgcatggaggctgcccgcacctgctcccgcctgctcacaccctccatccacctcatcagtggccatgctcatgtggttagccagaccgcagtgcaagtggtggcagatgtgcttagcaaactgctcgtagttgggataacagatcctgaccctgacattcgctactgtgtcttggcgtccctggacgagcgctttgatgcacacctggcccaggcggagaacttgcaggccttgtttgtggctctgaatgaccaggtgtttgagatccgggagctggccatctgcactgtgggccgactcagtagcatgaaccctgcctttgtcatgcctttcctgcgcaagatgctcatccagattttgacagagttggagcacagtgggattggaagaatcaaagagcagagtgcccgcatgctggggcacctggtctccaatgccccccgactcatccgcccctacatggagcctattctgaaggcattaattttgaaactgaaagatccagaccctgatccaaacccaggtgtgatcaataatgtcctggcaacaataggagaattggcacaggttagtggcctggaaatgaggaaatgggttgatgaactttttattatcatcatggacatgctccaggattcctctttgttggccaaaaggcaggtggctctgtggaccctgggacagttggtggccagcactggctatgtagtagagccctacaggaagtaccctactttgcttgaggtgctactgaattttctgaagactgagcagaaccagggtacacgcagagaggccatccgtgtgttagggcttttaggggctttggatccttacaagcacaaagtgaacattggcatgatagaccagtcccgggatgcctctgctgtcagcctgtcagaatccaagtcaagtcaggattcctctgactatagcactagtgaaatgctggtcaacatgggaaacttgcctctggatgagttctacccagctgtgtccatggtggccctgatgcggatcttccgagaccagtcactctctcatcatcacaccatggttgtccaggccatcaccttcatcttcaagtccctgggactcaaatgtgtgcagttcctgccccaggtcatgcccacgttccttaacgtcattcgagtctgtgatggggccatccgggaatttttgttccagcagctgggaatgttggtgtcctttgtgaagagccacatcagaccttatatggatgaaatagtcaccctcatgagagaattctgggtcatgaacacctcaattcagagcacgatcattcttctcattgagcaaattgtggtagctcttgggggtgaatttaagctctacctgccccagctgatcccacacatgctgcgtgtcttcatgcatgacaacagcccaggccgcattgtctctatcaagttactggctgcaatccagctgtttggcgccaacctggatgactacctgcatttactgctgcctcctattgttaagttgtttgatgcccctgaagctccactgccatctcgaaaggcagcgctagagactgtggaccgcctgacggagtccctggatttcactgactatgcctcccggatcattcaccctattgttcgaacactggaccagagcccagaactgcgctccacagccatggacacgctgtcttcacttgtttttcagctggggaagaagtaccaaattttcattccaatggtgaataaagttctggtgcgacaccgaatcaatcatcagcgctatgatgtgctcatctgcagaattgtcaagggatacacacttgctgatgaagaggaggatcctttgatttaccagcatcggatgcttaggagtggccaaggggatgcattggctagtggaccagtggaaacaggacccatgaagaaactgcacgtcagcaccatcaacctccaaaaggcctggggcgctgccaggagggtctccaaagatgactggctggaatggctgagacggctgagcctggagctgctgaaggactcatcatcgccctccctgcgctcctgctgggccctggcacaggcctacaacccgatggccagggatctcttcaatgctgcatttgtgtcctgctggtctgaactgaatgaagatcaacaggatgagctcatcagaagcatcgagttggccctcacctcacaagacatcgctgaagtcacacagaccctcttaaacttggctgaattcatggaacacagtgacaagggccccctgccactgagagatgacaatggcattgttctgctgggtgagagagctgccaagtgccgagcatatgccaaagcactacactacaaagaactggagttccagaaaggccccacccctgccattctagaatctctcatcagcattaataataagctacagcagccggaggcagcggccggagtgttagaatatgccatgaaacactttggagagctggagatccaggctacctggtatgagaaactgcacgagtgggaggatgcccttgtggcctatgacaagaaaatggacaccaacaaggacgacccagagctgatgctgggccgcatgcgctgcctcgaggccttgggggaatggggtcaactccaccagcagtgctgtgaaaagtggaccctggttaatgatgagacccaagccaagatggcccggatggctgctgcagctgcatggggtttaggtcagtgggacagcatggaagaatacacctgtatgatccctcgggacacccatgatggggcattttatagagctgtgctggcactgcatcaggacctcttctccttggcacaacagtgcattgacaaggccagggacctgctggatgctgaattaactgcgatggcaggagagagttacagtcgggcatatggggccatggtttcttgccacatgctgtccgagctggaggaggttatccagtacaaacttgtccccgagcgacgagagatcatccgccagatctggtgggagagactgcagggctgccagcgtatcgtagaggactggcagaaaatccttatggtgcggtcccttgtggtcagccctcatgaagacatgagaacctggctcaagtatgcaagcctgtgcggcaagagtggcaggctggctcttgctcataaaactttagtgttgctcctgggagttgatccgtctcggcaacttgaccatcctctgccaacagttcaccctcaggtgacctatgcctacatgaaaaacatgtggaagagtgcccgcaagatcgatgccttccagcacatgcagcattttgtccagaccatgcagcaacaggcccagcatgccatcgctactgaggaccagcagcataagcaggaactgcacaagctcatggcccgatgcttcctgaaacttggagagtggcagctgaatctacagggcatcaatgagagcacaatccccaaagtgctgcagtactacagcgccgccacagagcacgaccgcagctggtacaaggcctggcatgcgtgggcagtgatgaacttcgaagctgtgctacactacaaacatcagaaccaagcccgcgatgagaagaagaaactgcgtcatgccagcggggccaacatcaccaacgccaccactgccgccaccacggccgccactgccaccaccactgccagcaccgagggcagcaacagtgagagcgaggccgagagcaccgagaacagccccaccccatcgccgctgcagaagaaggtcactgaggatctgtccaaaaccctcctgatgtacacggtgcctgccgtccagggcttcttccgttccatctccttgtcacgaggcaacaacctccaggatacactcagagttctcaccttatggtttgattatggtcactggccagatgtcaatgaggccttagtggagggggtgaaagccatccagattgatacctggctacaggttatacctcagctcattgcaagaattgatacgcccagacccttggtgggacgtctcattcaccagcttctcacagacattggtcggtaccacccccaggccctcatctacccactgacagtggcttctaagtctaccacgacagcccggcacaatgcagccaacaagattctgaagaacatgtgtgagcacagcaacaccctggtccagcaggccatgatggtgagcgaggagctgatccgagtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaagcagctgcctcagctcacatccttagagctgcaatatgtttccccaaaacttctgatgtgccgggaccttgaattggctgtgccaggaacatatgaccccaaccagccaatcattcgcattcagtccatagcaccgtctttgcaagtcatcacatccaagcagaggccccggaaattgacacttatgggcagcaacggacatgagtttgttttccttctaaaaggccatgaagatctgcgccaggatgagcgtgtgatgcagctcttcggcctggttaacacccttctggccaatgacccaacatctcttcggaaaaacctcagcatccagagatacgctgtcatccctttatcgaccaactcgggcctcattggctgggttccccactgtgacacactgcacgccctcatccgggactacagggagaagaagaagatccttctcaacatcgagcatcgcatcatgttgcggatggctccggactatgaccacttgactctgatgcagaaggtggaggtgtttgagcatgccgtcaataatacagctggggacgacctggccaagctgctgtggctgaaaagccccagctccgaggtgtggtttgaccgaagaaccaattatacccgttctttagcggtcatgtcaatggttgggtatattttaggcctgggagatagacacccatccaacctgatgctggaccgtctgagtgggaagatcctgcacattgactttggggactgctttgaggttgctatgacccgagagaagtttccagagaagattccatttagactaacaagaatgttgaccaatgctatggaggttacaggcctggatggcaactacagaatcacatgccacacagtgatggaggtgctgcgagagcacaaggacagtgtcatggccgtgctggaagcctttgtctatgaccccttgctgaactggaggctgatggacacaaataccaaaggcaacaagcgatcccgaacgaggacggattcctactctgctggccagtcagtcgaaattttggacggtgtggaacttggagagccagcccataagaaaacggggaccacagtgccagaatctattcattctttcattggagacggtttggtgaaaccagaggccctaaataagaaagctatccagattattaacagggttcgagataagctcactggtcgggacttctctcatgatgacactttggatgttccaacgcaagttgagctgctcatcaaacaagcgacatcccatgaaaacctctgccagtgctatattggctggtgccctttctggtaa

SEQ ID NO: 2 - protein, 2549 aa, Homo sapiens
  PEPTIDE (1)..(2549): wild type mTOR
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 3 - DNA, 20 bp, Artificial Sequence
  forward primer
taggttacaggcctggatgg

SEQ ID NO: 4 - DNA, 20 bp, Artificial Sequence
  reverse primer
cttggcctcccaaaatgtta

SEQ ID NO: 5 - DNA, 21 bp, Artificial Sequence
  forward primer
tccaggctacctggtatgaga

SEQ ID NO: 6 - DNA, 20 bp, Artificial Sequence
  reverse primer
gccttcctttcaaatccaaa

SEQ ID NO: 7 - DNA, 23 bp, Artificial Sequence
  forward primer (C1483R)
ggcctcgaggcggcgcatgcggc

SEQ ID NO: 8 - DNA, 23 bp, Artificial Sequence
  reverse primer (C1483R)
gccgcatgcgccgcctcgaggcc

SEQ ID NO: 9 - DNA, 33 bp, Artificial Sequence
  forward primer (E2419G)
gtcatggccgtgctgggagcctttgtctatgac

SEQ ID NO: 10 - DNA, 33 bp, Artificial Sequence
  reverse primer (E2419G)
gtcatagacaaaggctcccagcacggccatgac

SEQ ID NO: 11 - DNA, 33 bp, Artificial Sequence
  forward primer (L2427Q)
gtctatgaccccttgcagaactggaggctgatg

SEQ ID NO: 12 - DNA, 33 bp, Artificial Sequence
  reverse primer (L2427Q)
catcagcctccagttctgcaaggggtcatagac

SEQ ID NO: 13 - DNA, 23 bp, Artificial Sequence
  forward primer (C1483Y)
gccgcatgcgctacctcgaggcc

SEQ ID NO: 14 - DNA, 23 bp, Artificial Sequence
  reverse primer (C1483Y)
ggcctcgaggtagcgcatgcggc

SEQ ID NO: 15 - DNA, 35 bp, Artificial Sequence
  forward primer (E2419K)
gtgtcatggccgtgctgaaagcctttgtctatgac

SEQ ID NO: 16 - DNA, 35 bp, Artificial Sequence
  reverse primer (E2419K)
gtcatagacaaaggctttcagcacggccatgacac

SEQ ID NO: 17 - DNA, 33 bp, Artificial Sequence
  forward primer (L2427P)
gtctatgaccccttgccgaactggaggctgatg

SEQ ID NO: 18 - DNA, 33 bp, Artificial Sequence
  reverse primer (L2427P)
catcagcctccagttcggcaaggggtcatagac

SEQ ID NO: 19 - DNA, 36 bp, Artificial Sequence
  annealing primer
aattccaattgcccgggcttaagatcgatacgcgta

SEQ ID NO: 20 - DNA, 36 bp, Artificial Sequence
  annealing primer
ccggtacgcgtatcgatcttaagcccgggcaattgg

SEQ ID NO: 21 - DNA, 50 bp, Artificial Sequence
  hmTOR-MfeI-flag-F
gatcacaattgtggccaccatggactacaaggacgacgatgacaagatgc

SEQ ID NO: 22 - DNA, 41 bp, Artificial Sequence
  hmTOR-MluI-R
tgatcaacgcgtttaccagaaagggcaccagccaatatagc